(19) United States
(12) Patent Application Publication
(10) Pub. No.: US 2014/0308199 A1
(43) Pub. Date: Oct. 16, 2014

(54) PROCESS FOR PRODUCING LIQUID FUEL FROM GAS STREAM COMPRISING CARBON DIOXIDE

(71) Applicant: ANTECY B.V., Hoevelaken (NL)

(72) Inventors: Paul O'CONNOR, HOEVELAKEN (NL); Jacobus Cornelis RASSER, AMSTERDAM (NL)

(21) Appl. No.: 14/316,855

(22) Filed: Jun. 27, 2014

Related U.S. Application Data
(63) Continuation of application No. PCT/EP2012/076456, filed on Dec. 20, 2012.
(60) Provisional application No. 61/580,677, filed on Dec. 28, 2011.

(51) Int. Cl. C07C 29/00

(52) U.S. Cl. CPC C07C 29/00
USPC 423/580.1; 568/840

(57) ABSTRACT

A process for producing liquid fuel and liquid water from a gas stream comprising carbon dioxide and water vapor. The gas stream can be ambient air. In the process, carbon dioxide and water vapor are adsorbed onto an adsorbent. The gases are desorbed from the adsorbent to create a reaction mixture for forming the liquid fuel, such as methanol. Excess water vapor is condensed to form liquid water, which can be used in a variety of ways.

### CROSS REFERENCE TO RELATED APPLICATIONS

This application is a continuation of PCT application number PCT/EP2012/076456 filed on 20 Dec. 2012, which claims priority from U.S. application No. 61/580,677 filed on 28 Dec. 2011. Both applications are hereby incorporated by reference in their entireties.

### BACKGROUND OF THE INVENTION

1. Field of the Invention

This application is a continuation of PCT application number PCT/EP2012/076456 filed on 20 Dec. 2012, which claims priority from U.S. application No. 61/580,677 filed on 28 Dec. 2011. Both applications are hereby incorporated by reference in their entireties.

The invention relates generally to a process for producing liquid fuel from a gas stream comprising carbon dioxide, and more particularly to a process for producing liquid fuel from and liquid water a gas stream comprising carbon dioxide and water vapor.

2. Description of the Related Art

Carbon dioxide is abundantly available in the Earth's atmosphere. In fact, its abundance has reached a point where it is believed to contribute to climate change. Atmospheric carbon dioxide in excess of historically recorded values can be considered a pollutant.

Industrial processes have been proposed for converting carbon dioxide from industrial sources, such as power plants, into liquid fuel, for example methanol. The proposed processes suffer from a number of drawbacks.

Firstly, such processes require gas streams that are, relative to ambient air, enriched in carbon dioxide. Such carbon dioxide gas streams are available in the form of flue gases of industrial plants that burn fossil or renewable fuels, such as refineries and power plants. Accordingly, the proposed processes require to be integrated with industrial plants.

Secondly, fuel gases generally are contaminated with other gases, such as nitrogen oxides and sulfur oxides. These gases are corrosive to the equipment needed in the carbon dioxide conversion process, and poisonous to catalysts used in the carbon dioxide conversion process. For these reasons flue gases require extensive (and expensive) scrubbing before they can be used in the proposed carbon dioxide conversion processes.

Thirdly, the proposed processes require hydrogen or water as a hydrogen source. Processes reliant on hydrogen are inherently expensive. Processes reliant on water as a hydrogen source compete with other human needs for this increasingly scarce resource.

Thus, there is a need for a process for producing liquid fuel from carbon dioxide recovered from a dilute gas stream. There is a particular need for such process that uses carbon dioxide and water vapor from ambient air.

### BRIEF SUMMARY OF THE INVENTION

The present invention addresses these problems by providing a process for producing liquid fuel and liquid water from a gas stream comprising carbon dioxide and water vapor, said process comprising the steps of;
a. Contacting the gas stream with one or more adsorbent materials, whereby carbon dioxide and water vapor are adsorbed to the adsorbent;
b. Desorbing carbon dioxide and water from the one or more adsorbent materials;
c. Reacting carbon dioxide and water to form a liquid fuel;
d. Condensing excess water from step b. to form liquid water.

### DETAILED DESCRIPTION OF THE INVENTION

The following is a detailed description of the invention. In its broadest aspect the invention provides a process for producing liquid fuel and liquid water from a gas stream comprising carbon dioxide and water vapor, said process comprising the steps of:
a. Contacting the gas stream with one or more adsorbent materials, whereby carbon dioxide and water vapor are adsorbed to the adsorbent;
b. Desorbing carbon dioxide and water from the one or more adsorbent materials;
c. Reacting carbon dioxide and water to form a liquid fuel;
d. Condensing excess water from step b. to form liquid water.

Thus, the present invention relates to a process for producing a liquid fuel from carbon dioxide and water vapor. By way of illustration, the liquid fuel can be methanol. The nominal reaction equation for the formation of methanol (CH₃OH) from carbon dioxide and water is:

2 CO₂+4 H₂O→2 CH₃OH+3 O₂

Similarly, for the formation of n-alkanes (general formula CₙH₂ₙ₊₂):

2n CO₂+(2n+2) H₂O→2CₙH₂ₙ₊₂+(n+1) O₂

As can be seen, the reaction for methanol requires carbon dioxide and water in a molar ratio of 1:2; the reaction for n-alkanes requires carbon dioxide and water in a molar ratio of n:(n+1), which for n-octane is about 0.9.

Both carbon dioxide and water vapor are taken from a gas stream, which can be ambient air. The amount of carbon dioxide in ambient air varies with time and location, but is generally about 400 ppm, or about 9 mmole per kg of dry air. The amount of water vapor in air is, of course, subject to wide fluctuations. By way of example, air having a temperature of 20 ° C. and a relative humidity of 50% contains about 7000 ppm water vapor, or about 400 mmole per kg. Under all but the most extreme conditions, ambient air contains water vapor and carbon dioxide in a molar ratio that well exceeds the 2:1 ratio required for the formation of methanol, or the n:(n+1) ratio required for the formation of n-alkanes.

It follows that if the reactants carbon dioxide and water are both recovered from a gas stream such as ambient air, it is highly feasible to recover more water than is needed for the reaction with carbon dioxide. An important aspect of the process of the present invention is that this excess water is condensed, so it is in a suitable form to be used in, for example, irrigation or cleaning, or, after an optional purification step, for food processing or as drinking water.

An important aspect of the process of the invention is the adsorption of carbon dioxide and water vapor from a gas stream. This can be accomplished, for example, by forcing a gas stream, such as ambient air, through a packed bed of adsorbent material. In general adsorption can take place at ambient temperature, except if the ambient temperature is below freezing.

In one embodiment carbon dioxide and water vapor are simultaneously adsorbed to a single bed of adsorbent material. In this embodiment water vapor and carbon dioxide compete for the same adsorbent sites. As explained above, water vapor is present in a significant molar excess, particularly under conditions of high absolute humidity. Therefore water vapor may crowd out carbon dioxide in the adsorption process, making the adsorption of carbon dioxide less efficient.

To avoid this crowding-out effect the adsorption can be carried out on two adsorbent materials, so that water is predominantly adsorbed onto the first adsorbent, and carbon dioxide is predominantly adsorbed onto the second adsorbent. Preferably the two adsorbent beds are placed in series, so that the gas stream is first contacted with the first adsorbent, and subsequently with the second adsorbent.

Preferably the adsorbent materials and the linear space velocity of the gas stream are selected so that the gas stream is substantially free of water vapor at the time of contacting the gas stream with the second adsorbent material.

The first and second adsorbent materials can be the same, or different. If the two adsorbent materials are different, each can be selected for optimum performance of its task. In other words, the first adsorbent can be selected to efficiently adsorb water vapor, while the second adsorbent can be selected to efficiently adsorb carbon dioxide.

As indicated above, the adsorption step requires creating a flow of air through a bed of the adsorbent material. The air flow can be created using mechanical means, such as a fan. Use of such mechanical means has the advantage that a desired air flow can be created at any desired time. A serious downside of this approach is that the mechanical means require an energy input.

An alternate way of creating the desired air flow makes use of naturally occurring air flows, such as wind. For example, the adsorbent bed can be placed in a tubular reactor with two open ends. A first open end is positioned to face the direction of the wind. The wind forces an air flow through the adsorbent bed. The air flow can be increased by placing a conical collector in front of the open end of the tubular reactor. The conical collector has a first diameter d₁ corresponding to the diameter of the tubular reactor, and a second diameter d₂ positioned upwind from the tubular reactor. The conical collector increases the velocity of the air flow by a factor corresponding to the square of the ratio d₂/d₁, i.e., a factor (d₁/d₂)².

The tubular reactor can be made to automatically orient itself relative to the direction of the wind by positioning the reactor on a pivotable platform. The platform has a point of rotation about a substantially vertical axis of rotation. The point of rotation is placed asymmetrically, so that there is a first portion of the reactor having length L₁ extending from the point of rotation, and a second portion having length L₂, whereby L₂>L₁. The tubular reactor automatically positions itself so that the opening of the first portion faces the direction of the wind. The second portion can be provided with one or more vanes to aid in the automatic positioning of the reactor. In yet another embodiment the air flow is created by movement of a vehicle. For example, a tubular reactor can be placed on the roof of a rail car, or on top of the trailer of a tractor/trailer combination. In a passenger car a reactor can be placed behind the front grille, so as not to interfere with the overall appearance of the vehicle. The slight increase in fuel consumption caused by the air resistance of the tube is far less than the energy that would be required for creating a comparable air flow through the reactor by means of a fan.

Although thus far the process has been discussed in terms of adsorbing carbon dioxide from ambient air, it will be understood that the process can also be used for adsorbing water vapor and carbon dioxide from the flue gas of an apparatus in which combustion of a carbon-based fuel takes place. The carbon-based fuel can be a fossil fuel, or a renewable fuel, or a combination of a fossil fuel and a renewable fuel.

Using a flue gas as the source of water vapor and carbon dioxide offers the advantage that both are present at increased concentrations, in comparison to ambient air. This embodiment also has significant disadvantages, however. The corrosive and catalyst-poisoning effects of contaminants present in flue gas have been discussed above. In addition, the molar ratio of water vapor and carbon dioxide in a flue gas are determined by the carbon/hydrogen ratio of the fuel that was burned in creating the flue gas. It should be clear that no excess water is present in a flue gas. In addition, flue gas needs to be cooled in order to be adsorbed onto the adsorbent material. The necessary cooling results in condensation of a significant portion of the water vapor present in the flue gas, as a result of which the water vapor/carbon dioxide molar ratio of the cooled flue gas is below the minimum required for the process.

Thus, it has surprisingly been found that ambient air is in fact far more suitable for use in the process of the present invention than is a flue gas. The use of ambient air offers the additional advantage that it can be carried out on a small scale. An adsorption unit the size of a domestic refrigerator is large enough to provide a family home with the amount of reactants (water and carbon dioxide) needed to produce the amount of liquid fuel to supply the necessary energy. In addition, such a unit supplies significant amounts of liquid water, resulting in further cost savings that help defray the cost of the installation.

As described in the prior art, the reaction of carbon dioxide and water vapor (via hydrogen) to produce liquid fuel (such as methanol) can be carried out catalytically at relatively modest temperatures, i.e. in the range of 200 to 300° C. See, for example, M. Saito et al. Applied Catalysis A: General 138 (1996) 311-318.

Both carbon dioxide are desorbed from the adsorbent (or respective adsorbents) by increasing the temperature of the adsorbent. Preferably the temperature for desorption is close to the reaction temperature, so that no excess energy is needed for the desorption step. In other words, the desorption step preferably takes place at a temperature in the range of 200 to 300° C.

Examples of suitable adsorbents include oxides of alkali metals, alkaline earth metals, and non-noble transition metals, in particular TiO₂; K₂O; MgO; Al₂O₃; ZnO; FeₓOy; BaO; CaO; MnₓO_{y}; CuO; and mixtures thereof. These materials can be used as the sole adsorbent material, or as the second adsorbent material in the two-adsorbents embodiment.

In the two-adsorbents embodiment the first adsorbent's role is the adsorption of water vapor. Preferably the first adsorbent is specifically dedicated to this task, and selected from the large number of well known drying agents. Examples include zeolites, and desiccants such as CaCl₂.

Many modifications in addition to those described above may be made to the structures and techniques described herein without departing from the spirit and scope of the invention. Accordingly, although specific embodiments have been described, these are examples only and are not limiting upon the scope of the invention.

What is claimed is:

1. A process for producing liquid fuel and liquid water from a gas stream comprising carbon dioxide and water vapor, said process comprising the steps of:
a. Contacting the gas stream with one or more adsorbent materials, whereby carbon dioxide and water vapor are adsorbed to the adsorbent;
b. Desorbing carbon dioxide and water from the one or more adsorbent materials;
c. Reacting carbon dioxide and water to form a liquid fuel;
d. Condensing excess water from step b. to form liquid water.

2. The process of claim 1, wherein carbon dioxide and water vapor are adsorbed to one adsorbent material.

3. The process of claim 1, wherein water vapor is predominantly adsorbed to a first adsorbent material, and carbon dioxide is predominantly adsorbed to a second adsorbent material.

4. The process of claim 3, wherein the gas stream is first contacted with the first adsorbent material, and subsequently contacted with the second adsorbent material.

5. The process of claim 4, wherein the gas stream is substantially free of water vapor at the time of contacting the gas stream with the second adsorbent material.

6. The process of claim 1, wherein step a. comprises forcing a flow of air through a bed of adsorbent material.

7. The process of claim 6, wherein the flow of air is created by mechanical means.

8. The process of claim 6, wherein the flow of air is caused by wind.

9. The process of claim 6, wherein the flow of air is caused by movement of a vehicle.

10. The process of claim 1, wherein the gas stream comprises a flue gas of an apparatus in which combustion of a carbon-based fuel takes place.

11. The process of claim 10, wherein the carbon-based fuel comprises a renewable fuel.

12. The process of claim 11, wherein the renewable fuel comprises the liquid fuel produced by the process of claim 1.

13. The process of claim 10, wherein the carbon-based fuel comprises a fossil fuel.

14. The process of claim 1, wherein the gas stream comprises ambient air.

15. The process of claim 1, wherein the one or more adsorbent materials are selected from the group consisting of TiO₂; K₂O; MgO; Al₂O₃; ZnO; FeₓOy; BaO; CaO; MnₓO_{y}; CuO; and mixtures thereof.

16. The process of claim 4, wherein the first adsorbent material is selected from the group consisting of zeolites, desiccants, and mixtures thereof.

17. The process of claim 4, wherein the second adsorbent material is selected from the group consisting of TiO₂; K₂O; MgO; Al₂O₃; ZnO; FeₓOy; BaO; CaO; MnₓO_{y}; CuO; and mixtures thereof.

18. The process of claim 1, wherein the liquid fuel comprises methanol.

19. The process of claim 1, wherein the liquid water is used for domestic purposes.

20. The process of claim 1, wherein the liquid water is used for agricultural purposes.
